(19) United States
(12) Patent Application Publication
(10) Pub. No.: US 2008/0305042 A1
(43) Pub. Date: Dec. 11, 2008

(54) Radiolabelling Method Using Polymers

(76) Inventors: Michel Gacek, Hovik (NO); Hanno Priebe, Oslo (NO); Nigel John Osborn, Bucks (GB)

Correspondence Address: GE HEALTHCARE, INC., IP DEPARTMENT, 101 CARNEGIE CENTER, PRINCETON, NJ, 08540-6231, US

(21) Appl. No.: 12/095,923

(22) PCT Filed: Dec. 5, 2006
(86) PCT No.: PCT/GB2006/004533

§ 371 Date: Jun. 3, 2008

(30) Foreign Application Priority Data
Dec. 6, 2005 (GB) 0524851.3

(51) Int. Cl. A61K 51/06; A61K 51/08

(52) U.S. Cl. 424/1.89; 424/1.65; 424/1.81

(57) ABSTRACT

The present invention provides a method for the preparation of radioisotopically-labelled imaging agent compositions. The method uses precursors which are bound to soluble polymers, so that the radiolabelling reaction is carried out in solution. Also described are radiopharmaceutical compositions, automated versions of the radiolabelling method and disposable cassettes for use in the automated method.

### FIELD OF THE INVENTION

The present invention provides a method for the preparation of radioisotopically-labelled imaging agent compositions. The method uses precursors which are bound to soluble polymers, so that the radiolabelling reaction is carried out in solution. Also described are radiopharmaceutical compositions, automated versions of the radiolabelling method and disposable cassettes for use in the automated method.

### BACKGROUND TO THE INVENTION

In the synthesis of radiopharmaceuticals such as 2-[¹⁸F]-fluoro-2-deoxyglucose (¹⁸F-FDG), the yield of the final product is limited by the short half-life of the radioisotope (110 mins for ¹⁸F). Hence, the synthesis time is of crucial importance to the yield. The radiolabelling reaction is typically based on the reaction of a non-radioactive precursor with a supply of the radioisotope, wherein the precursor is present in large chemical excess. When such radiolabelling reactions are performed in solution, the consequence is that the radiopharmaceutical product also contains the excess non-radioactive precursor molecule (or a deprotected variant thereof). The need for purification is especially important where the precursor molecule has significant toxicity or could impact on the efficacy of the radiopharmaceutical by saturating or competing with available target binding sites in vivo. The problem is that conventional chemical separation techniques such as high performance liquid chromatography (HPLC) are generally too time-consuming to be applicable to such radiosyntheses.

It is known in the art to use polymer-supported reagents in the solid-phase synthesis of radioisotopically-labelled imaging agents to solve this problem. This methodology involves binding the non-radioactive precursor for radiolabelling to a suitable solid support, with the radioisotope such as ¹⁸F-fluoride displacing the radiotracer, leaving excess precursor bound to the solid support. Examples of this are WO 04/056399 (Solid-phase Fluorination of Benzothiazoles); WO 04/056400 (Solid-phase Fluorination of Uracil and Cytosine); WO 04/056725 (Solid-phase Preparation of ¹⁸F-Labelled Amino Acids) and WO 03/002157 (Solid-phase Nucleophilic Fluorination). The primary advantage of the attachment of one of the reaction components to an insoluble polymer is that removal of the radiopharmaceutical product from the reaction mixture is easily accomplished, because one or more of the starting materials remains bound to the solid phase, and the product is generated in solution. Unfortunately, the heterogeneous reaction conditions occasioned by the use of insoluble polymers sometimes complicates the transfer of the corresponding non-radioactive solution-phase chemical methodology to radiosyntheses. This can result in low or zero yields for the solid-phase process, whereas the corresponding solution phase chemistry is viable. Since the radioisotope must diffuse into the pores of the resin, react with the precursor and the resultant radiolabelled product must diffuse out again, there is reduced efficiency of mixing and/or accessibility of reactive sites relative to solution phase methodology. Consequently, the reaction kinetics are slower.

There is therefore a need for improved preparation methods for radioisotopically-labelled imaging agent compositions.

### THE PRESENT INVENTION

Prior art solid polymer supports are normally highly cross-linked, rigid macroporous resins. When functionalised, such polymers have approximately 3% or less of the functional groups located on the surface of the beads, the rest being inside. For radiosynthesis precursors this translates to ca. 97% of the conjugated precursor being likely to be contained within the pores of the resin. The present inventors believe that, where solid-phase diffusion rates limit ingress of the radioisotope this is likely to manifest itself in terms of lower radiochemical purity (RCP), and where diffusion rates limit diffusion out of the pores this will result in lowered recovery, ie. yield. Additionally the problem of getting efficient reaction is compounded by the relatively poor swelling ability of polystyrene-based solid supports in the polar organic solvents, such as acetonitrile, generally used for radiofluorination reactions.

The present invention provides a method of preparation of radioisotopically-labelled imaging agent compositions wherein the non-radioactive precursor is conjugated to a soluble macromolecule for subsequent or concurrent displacement with the radioisotope. Such a soluble polymer has a more open structure, so that the reaction is no longer limited by the diffusion rate of the radioisotope into and out of the pores of the resin.

The present method represents a middle point between the kinetic advantages of using entirely solution phase chemistry with its consequent poor purity profile, and solid phase chemistry, with slower kinetics but better purity. The present invention uses polymers which are macromolecules soluble either in organic or aqueous solution. This is essentially a surrogate resin, where all subsequent radiolabelling is carried out in the solution phase rather than the solid phase. The subsequent purification of the desired radioactive imaging agent from the macromolecule can be achieved either chromatographically or through precipitation/extraction. The soluble polymer approach of the present invention is expected to be particularly useful for reactions in which the precursor is sterically bulky, and hence would be less able to access the internal surfaces of the resin.

### DETAILED DESCRIPTION OF THE INVENTION

In a first aspect, the present invention provides a method of preparation of a radioisotopically-labelled imaging agent composition which comprises the process of:
(i) provision of a conjugate which comprises a precursor to said imaging agent covalently bound to a polymer, wherein said precursor has at least one group (X) which provides a reactive site for radiolabelling;
(ii) reaction in a suitable solvent of a solution of the conjugate from step (i) with a chemical form of the radioisotope suitable for reaction with X to give a solution of the radiolabelled precursor bound to said polymer;
(iii) cleavage of the radiolabelled precursor product of step (ii) from the polymer;
(iv) separation of the cleaved radiolabelled precursor product of step (iii) from the polymer and optionally from other reaction products of steps (ii) and (iii);
(v) when the separated radiolabelled precursor product of step (iv) is already in a biocompatible carrier medium, it is used directly in step (vi), otherwise the product of step (iv) is either dissolved in a biocompatible carrier medium or the solvent of step (iv) is removed in part or in full, and replaced with a biocompatible carrier medium;
(vi) optionally carrying out one or more of the following additional processes on the product of step (v): purification; pH adjustment; dilution or concentration; solvent removal and re-dissolution in a biocompatible solvent; to give the desired imaging agent composition.

By the term “imaging agent” is meant a compound suitable for administration to the mammalian body, especially the human body, for use in in vivo imaging. The imaging agents of the present invention comprise a biological targeting molecule (“tracer”) which is radioisotopically-labelled. By the term “biological targeting molecule” or “tracer” is meant: 3-100 mer peptides or peptide analogues which may be linear peptides or cyclic peptides or combinations thereof; amino acids, including unnatural amino acids; enzyme substrates, agonists, antagonists or inhibitors; synthetic receptor-binding compounds; oligonucleotides, oligo-DNA or oligo-RNA fragments; nucleosides or hypoxia-localising nitroaromatic compounds such as nitroimidazoles.

By the term “cyclic peptide” is meant a sequence of 5 to 15 amino acids in which the two terminal amino acids are bonded together by a covalent bond which may be a peptide or disulphide bond or a synthetic non-peptide bond such as a thioether, phosphodiester, disiloxane or urethane bond. By the term “amino acid” is meant an L- or D-amino acid, amino acid analogue or amino acid mimetic which may be optically pure, i.e. a single enantiomer and hence chiral, or a mixture of enantiomers. Preferably the amino acids of the present invention are optically pure. By the term “amino acid mimetic” is meant synthetic analogues of naturally occurring amino acids which are isosteres, i.e. have been designed to mimic the steric and electronic structure of the natural compound. Such isosteres are well known to those skilled in the art and include but are not limited to depsipeptides, retro-inverso peptides, thioamides, cycloalkanes or 1,5-disubstituted tetrazoles [see M. Goodman, Biopolymers, 24, 137, (1985)].

Suitable peptides for use in the present invention include:
somatostatin, octreotide and analogues,
peptides which bind to the ST receptor, where ST refers to the heat-stable toxin produced by E. coli and other micro-organisms;
laminin fragments eg. YIGSR, PDSGR, IKVAV, LRE and KCQAGTFALRGDPQG,
N-formyl peptides for targeting sites of leucocyte accumulation,
Platelet factor 4 (PF4) and fragments thereof,
RGD (Arg-Gly-Asp)-containing peptides, which may eg. target angiogenesis [R. Pasqualini et al., Nat. Biotechnol. 1997 June; 15(6):542-6]; [E. Ruoslahti, Kidney Int. 1997 May; 51(5):1413-7].
peptide fragments of α₂-antiplasmin, fibronectin or beta-casein, fibrinogen or thrombospondin. The amino acid sequences of α₂-antiplasmin, fibronectin, beta-casein, fibrinogen and thrombospondin can be found in the following references: α₂-antiplasmin precursor [M. Tone et al., J.Biochem, 102, 1033, (1987)]; beta-casein [L. Hansson et al, Gene, 139, 193, (1994)]; fibronectin [A. Gutman et al, FEBS Lett., 207, 145, (1996)]; thrombospondin-1 precursor [V. Dixit et al, Proc. Natl. Acad. Sci., USA, 83, 5449, (1986)]; R. F. Doolittle, Ann. Rev. Biochem., 53, 195, (1984);
peptides which are substrates or inhibitors of angiotensin, such as: angiotensin II Asp-Arg-Val-Tyr-Ile-His-Pro-Phe (E. C. Jorgensen et al, J. Med. Chem., 1979, Vol 22, 9, 1038-1044)
[Sar, Ile] Angiotensin II: Sar-Arg-Val-Tyr-Ile-His-Pro-Ile (R. K. Turker et al., Science, 1972, 177, 1203).
Angiotensin I: Asp-Arg-Val-Tyr-Ile-His-Pro-Phe-His-Leu.

Preferably the peptides of the present invention comprise antiplasmin or angiotensin II peptides. Antiplasmin peptides comprise an amino acid sequence taken from the N-terminus of:

(i) α₂-antiplasmin,
i.e. NH₂-Asn-Gln-Glu-Gln-Val-Ser-Pro-Leu-Thr-Leu-Thr-Leu-Leu-Lys-OH or variants of this in which one or more amino acids have been exchanged, added or removed such as:

### NH₂-Asn-Gln-Glu-Gln-Val-Ser-Pro-Leu-Thr-Leu-Thr-Leu-Leu-Lys-Gly-OH,

### NH₂-Asn-Gln-Glu-Ala-Val-Ser-Pro-Leu-Thr-Leu-Thr-Leu-Leu-Lys-Gly-OH,

### NH₂-Asn-Gln-Glu-Gln-Val-Gly-OH; or

(ii) casein
ie. Ac-Leu-Gly-Pro-Gly-Gln-Ser-Lys-Val-Ile-Gly.

Synthetic peptides of the present invention may be obtained by conventional solid phase synthesis, as described in P. Lloyd-Williams, F. Albericio and E. Girald; Chemical Approaches to the Synthesis of Peptides and Proteins, CRC Press, 1997.

Suitable enzyme substrates, agonists antagonists or inhibitors include glucose and glucose analogues such as fluorodeoxyglucose (FDG); fatty acids, or elastase, angiotensin II or metalloproteinase inhibitors. A preferred non-peptide angiotensin II antagonist is Losartan.

Suitable synthetic receptor-binding compounds include estradiol, estrogen, progestin, progesterone and other steroid hormones; ligands for the dopamine D-1 or D-2 receptor, such as dihydroxyphenylalanine (DOPA) or dopamine transporter such as tropanes; and ligands for the serotonin receptor, such as Altanaserine which binds to the 5-HT2A serotonin receptor.

The biological targeting molecule is preferably of molecular weight of less than 5000, most preferably less than 4000, ideally less than 3000. Preferred biological targeting moieties are 3-20 mer peptides or enzyme substrates, enzyme antagonists or enzyme inhibitors.

By the term “radioisotopically-labelled” means that either a functional group of the tracer comprises the radioisotope, or the radioisotope is attached to the tracer as an additional species. When a functional group comprises the radioisotope, this means that the radioisotope forms part of the chemical structure of the tracer, and is a radioactive isotope present at a level significantly above the natural abundance level of said isotope. Such elevated or enriched levels of isotope are suitably at least 5 times, preferably at least 10 times, most preferably at least 20 times; and ideally either at least 50 times the natural abundance level of the isotope in question, or present at a level where the level of enrichment of the isotope in question is 90 to 100% Examples of such functional groups include CH₃ groups with elevated levels of ¹¹C and fluoroalkyl groups with elevated levels of ¹⁸F, such that the radioisotope is present as the isotopically labelled ¹¹C or ¹⁸F atom within the chemical structure.

When the radioisotope is attached as an additional species, this could be an attached metal complex of a radiometal, an ¹⁸F-substituted alkyl group in place of an unsubstituted alkyl group of the tracer, or a radioiodine-bearing aryl group in place of an unsubstituted aryl group of the tracer.

Suitable radioisotopes of the present invention may be detected in vivo either external to the mammalian body or via use of detectors designed for use in vivo, such as intravascular or radiation detectors designed for intra-operative use. The radioisotope is suitably chosen from:
(i) a radioactive metal ion;
(ii) a gamma-emitting radioactive halogen;
(iii) a positron-emitting radioactive non-metal;
(iv) a β-emitter suitable for intravascular detection.

Preferred radioisotopes are those which can be detected externally in a non-invasive manner following administration in vivo. Most preferred radioisotopes are chosen from: radioactive metal ions, gamma-emitting radioactive halogens or positron-emitting radioactive non-metals, particularly those suitable for imaging using SPECT or PET. Most especially preferred radioisotopes are positron emitters, suitable for PET imaging.

When the radioisotope is a radioactive metal ion, ie. a radiometal, suitable radiometals can be either positron emitters such as ⁶⁴Cu, ⁴⁸V, ⁵²Fe, ⁵⁵Co, ^{94m}Tc or ⁶⁸Ga; γ-emitters such as ^{99m}Tc, ¹¹¹In, ^{113m}In, or ⁶⁷Ga. Preferred radiometals are ^{99m}Tc, ⁶⁴Cu, ⁶⁸Ga and ¹¹¹In.

When the radioisotope is a gamma-emitting radioactive halogen, the radiohalogen is suitably chosen from ¹²³I, ¹³¹I or ⁷⁷Br. A preferred gamma-emitting radioactive halogen is ¹²³I.

When the radioisotope is a positron-emitting radioactive non-metal, suitable such positron emitters include: ¹¹C, ¹³N, ¹⁵O, ¹⁷F, ¹⁸F, ⁷⁵Br, ⁷⁶ Br or ¹²⁴I. Preferred positron-emitting radioactive non-metals are ¹¹C, ¹³N, ¹⁸F and ¹²⁴I, especially ¹¹C and ¹⁸F, most especially ¹⁸F.

When the radioisotope is a β-emitter suitable for intravascular detection, suitable such β-emitters include the radiometals ⁶⁷Cu, ⁸⁹Sr, ⁹⁰Y, ¹⁵³Sm, ¹⁸⁶Re, ¹⁸⁸Re or ¹⁹²Ir, and the non-metals ³²P, ³³P, ³⁸S, ³⁸Cl, ³⁹Cl, ⁸²Br and ⁸³Br.

The radioisotopes ³H, ¹⁴C and ¹²⁵I are least preferred radioisotopes for use in the present invention.

Suitable “conjugates” of the present invention comprise a precursor to the imaging agent covalently bound to a polymer. This means that the precursor is covalently bound to the biological targeting molecule (“tracer”).

By the term “precursor” is meant a non-radioactive derivative of the tracer, designed so that chemical reaction with a convenient chemical form of the radioisotope occurs site-specifically; can be conducted in the minimum number of steps (ideally a single step) and without the need for significant purification (ideally no further purification), to give the desired radioisotopically-labelled imaging agent product. Suitable precursors incorporate a group (X) which provides a reactive site for site-specific radiolabelling. X is covalently bonded to the precursor and is designed so that chemical reaction to introduce the radioisotope occurs specifically at X. Such precursors are synthetic and can conveniently be obtained in good chemical purity. The “precursor” may optionally comprise one or more protecting groups (P^{GP}) for certain functional groups of the biological targeting molecule. Suitable precursors are described in more detail below.

By the term “protecting group” (P^{GP}) is meant a group which inhibits or suppresses undesirable chemical reactions, but which is designed to be sufficiently reactive that it may be cleaved from the functional group in question under mild enough conditions that do not modify the rest of the molecule. After deprotection the desired product is obtained. Protecting groups are well known to those skilled in the art and are suitably chosen from, for amine groups: Boc (where Boc is tert-butyloxycarbonyl), Fmoc (where Fmoc is fluorenylmethoxycarbonyl), trifluoroacetyl, allyloxycarbonyl, Dde [i.e. 1-(4,4-dimethyl-2,6-dioxocyclohexylidene)ethyl] or Npys (i.e. 3-nitro-2-pyridine sulfenyl); and for carboxyl groups: methyl ester, tert-butyl ester or benzyl ester. For hydroxyl groups, suitable protecting groups are: methyl, ethyl or tert-butyl; alkoxymethyl or alkoxyethyl; benzyl; acetyl; benzoyl; trityl (Trt) or trialkylsilyl such as tetrabutyldimethylsilyl. For thiol groups, suitable protecting groups are: trityl and 4-methoxybenzyl. The use of further protecting groups are described in ‘Protective Groups in Organic Synthesis’, Theorodora W. Greene and Peter G. M. Wuts, (Third Edition, John Wiley & Sons, 1999).

When the radioisotope of the present invention is non-metallic, preferred precursors are those which comprise an X group which either undergoes electrophilic or nucleophilic halogenation or undergoes condensation with a labelled aldehyde or ketone. Examples of the first category are:
(a) organometallic derivatives such as a trialkylstannane (eg. trimethylstannyl or tributylstannyl substituents), or a trialkylsilane (eg. a trimethylsilyl substituent) or an organoboron compound (eg. boronate esters or organotrifluoroborates);
(b) a non-radioactive aryl bromide or iodide for halogen exchange or alkyl or aryl tosylate, mesylate or triflate for nucleophilic radiohalogenation;
(c) aromatic rings activated towards electrophilic iodination (eg. phenols) and aromatic rings activated towards nucleophilic radiohalogenation (eg. aryl iodonium salt aryl diazonium, aryl trialkylammonium salts or nitroaryl derivatives).

For such non-metallic radioisotopes, preferred X groups are chosen from: a non-radioactive halogen atom such as an aryl iodide or bromide (to permit radioiodine exchange); an activated aryl ring (e.g. a phenol group); an organometallic precursor compound (eg. trialkyltin, trialkylsilyl or organoboron compound); or an organic precursor such as triazenes or a good leaving group for nucleophilic substitution such as an iodonium salt. Most preferred X groups are activated aryl rings; organometallic compounds (eg. trialkyltin, trialkylsilyl or organoboron compound); or an organic precursor such as triazenes or a good leaving group for nucleophilic substitution.

Precursors and hence suitable X groups and methods of introducing radiohalogens into organic molecules are described by Bolton [J. Lab.Comp.Radiopharm., 45, 485-528 (2002)]. Precursors and methods of introducing radioiodine into proteins are described by Wilbur [Bioconj.Chem., 3(6), 433-470 (1992)]. Suitable boronate ester organoboron compounds and their preparation are described by Kabalaka et al [Nucl.Med.Biol., 29, 841-843 (2002) and 30, 369-373 (2003)]. Suitable organotrifluoroborates and their preparation are described by Kabalaka et al [Nucl.Med.Biol., 31, 935-938 (2004)].

Examples of suitable precursor aryl groups to which radioactive halogens, especially iodine can be attached are given below:

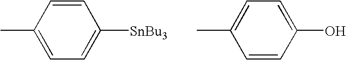

Both contain substituents which permit facile radioiodine substitution onto the aromatic ring. Alternative substituents containing radioactive iodine can be synthesised by direct iodination via radiohalogen exchange, e.g.

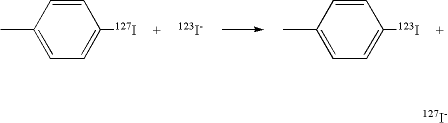

When the radioisotope comprises a radioiodine atom, it is preferably attached to the tracer via a direct covalent bond to an aromatic ring such as a benzene ring, or a vinyl group since it is known that iodine atoms bound to saturated aliphatic systems are prone to in vivo metabolism and hence loss of the radioiodine.

When the radioisotope comprises a radioactive isotope of fluorine (eg. ¹⁸F), the radioisotopic labelling may be carried out via direct labelling using the reaction of ¹⁸F-fluoride with a suitable precursor having a good leaving group, such as an alkyl bromide, alkyl mesylate or alkyl tosylate. ¹⁸F can also be introduced by N-alkylation of amine precursors with alkylating agents such as ¹⁸F(CH₂)₃OMs (where Ms is mesylate) to give N—(CH₂)₃¹⁸F, or O-alkylation of hydroxyl groups with ¹⁸F(CH₂)₃OMs or ¹⁸F(CH₂)₃Br. ¹⁸F can also be introduced by alkylation of N-haloacetyl groups with a ¹⁸F(CH₂)₃OH reactant, to give —NH(CO)CH₂O(CH₂)₃ ¹⁸F derivatives. For aryl systems, ¹⁸F-fluoride nucleophilic displacement from an aryl diazonium salt, aryl nitro compound or an aryl quaternary ammonium salt are possible routes to aryl-¹⁸F derivatives.

Primary amine-containing tracers can also be labelled with ¹⁸F by reductive amination, eg: using ¹⁸F—C₆H₄—CHO as taught by Kahn et al [J. Lab.Comp.Radiopharm. 45, 1045-1053 (2002)] and Borch et al [J. Am. Chem. Soc. 93, 2897 (1971)]. This approach can also usefully be applied where X is an aryl primary amine and comprises eg. phenyl-NH₂ or phenyl-CH₂NH₂ groups.

Amine-containing tracers can also be labelled with ¹⁸F by reaction with ¹⁸F-labelled active esters such as:

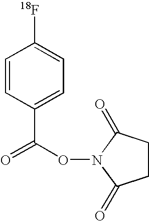

to give amide bond linked products. The N-hydroxysuccinimide ester shown and its use to label peptides is taught by Vaidyanathan et al [Nucl.Med.Biol., 19(3), 275-281 (1992)] and Johnstrom et al [Clin.Sci., 103 (Suppl. 48), 45-85 (2002)]. Further details of synthetic routes to ¹⁸F-labelled derivatives are described by Bolton,

### J. Lab.Comp.Radiophamm., 45, 485-528 (2002).

Introduction of PET radioisotope labels can also be achieved by O-alkylation of hydroxyl groups with triflate derivatives such as ¹¹CH₃OSO₂CF₃ as taught by Fei et al [J.Lab.Comp.Radiopharm., 46, 343-351 (2003)], or Zheng et al [Nucl.Med.Biol., 30, 753-760 (2003)], or the ¹⁸F O-alkylating reagents described above. ¹¹C PET radiolabels can also be introduced by use of the above triflate derivative to alkylate phenolic hydroxyl groups as taught by Zheng et al [Nucl. Med Biol., 31, 77-85 (2004)]. Further methods of labelling with ¹¹C are taught by Antoni et al [Chapter 5 pages 141-194 in “Handbook of Radiopharmaceuticals”, M. J. Welch and C. S. Redvanly (Eds.), Wiley (2003)].

When the radioisotope is a radiometal, i.e. comprises a metal ion, the metal ion is present as a metal complex. By the term “metal complex” is meant a coordination complex of the metal ion with one or more ligands. It is strongly preferred that the metal complex is “resistant to transchelation”, ie. does not readily undergo ligand exchange with other potentially competing ligands for the metal coordination sites. Potentially competing ligands include other excipients in the imaging agent composition in vitro (eg. radioprotectants or antimicrobial preservatives used in the preparation), or endogenous compounds in vivo (eg. glutathione, transferrin or plasma proteins).

The metal complexes of the present invention are derived from conjugates wherein the precursor comprises a metal complexing ligand, as described below.

Suitable ligands for use in the present invention which form metal complexes resistant to transchelation include: chelating agents, where 2-6, preferably 2-4, metal donor atoms are arranged such that 5- or 6-membered chelate rings result (by having a non-coordinating backbone of either carbon atoms or non-coordinating heteroatoms linking the metal donor atoms); or monodentate ligands which comprise donor atoms which bind strongly to the metal ion, such as isonitriles, phosphines or diazenides. Examples of donor atom types which bind well to metals as part of chelating agents are: amines, thiols, amides, oximes and phosphines. Phosphines form such strong metal complexes that even monodentate or bidentate phosphines form suitable metal complexes. The linear geometry of isonitriles and diazenides is such that they do not lend themselves readily to incorporation into chelating agents, and are hence typically used as monodentate ligands. Examples of suitable isonitriles include simple alkyl isonitriles such as tert-butylisonitrile, and ether-substituted isonitriles such as mibi (i.e. 1-isocyano-2-methoxy-2-methylpropane). Examples of suitable phosphines include Tetrofosmin, and monodentate phosphines such as tris(3-methoxypropyl)phosphine. Examples of suitable diazenides include the HYNIC series of ligands i.e. hydrazine-substituted pyridines or nicotinamides.

Examples of suitable chelating agents for technetium (^{99m}TC or ^{94m}Tc), copper (⁶⁴Cu or ⁶⁷Cu), vanadium (eg. ⁴⁸V), iron (eg. ⁵²Fe), or cobalt (eg. ⁵⁵Co) which form metal complexes resistant to transchelation include, but are not limited to:

(i) diaminedioximes of formula:

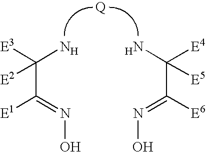

where E¹-E⁶ are each independently an R′ group;
each R′ is H or C₁₋₁₀ alkyl, C₃₋₁₀ alkylaryl, C₂₋₁₀ alkoxyalkyl, C₁₋₁₀ hydroxyalkyl, C₁₋₁₀ fluoroalkyl, C₂₋₁₀ carboxyalkyl or C₁₋₁₀ aminoalkyl, or two or more R′ groups together with the atoms to which they are attached form a carbocyclic, heterocyclic, saturated or unsaturated ring, and wherein one or more of the R′ groups is conjugated to the biological targeting molecule or tracer;
and Q is a bridging group of formula -(J)_{f}-;
where f is 3, 4 or 5 and each J is independently —O—, —NR′— or —C(R′)₂— provided that -(J)_{f}- contains a maximum of one J group which is —O— or —NR′—.

Preferred Q groups are as follows:

Q=—(CH₂)(CHR′)(CH₂)— ie. propyleneimine oxime or PnAO derivatives;
Q=—(CH₂)₂(CHR′)(CH₂)₂— ie. pentyleneamine oxime or PentAO derivatives;

### Q=—(CH₂)₂NR′(CH₂)₂—.

E¹ to E⁶ are preferably chosen from: C₁₋₃ alkyl, alkylaryl alkoxyalkyl, hydroxyalkyl, fluoroalkyl, carboxyalkyl or aminoalkyl. Most preferably, each E¹ to E⁶ group is CH₃.

The biological targeting molecule or tracer is preferably conjugated at either the E¹ or E⁶ R′ group, or an R′ group of the Q moiety. Most preferably, the tracer is conjugated to an R′ group of the Q moiety. When the tracer is conjugated to an R′ group of the Q moiety, the R′ group is preferably at the bridgehead position. In that case, Q is preferably —(CH₂)(CHR′)(CH₂)—, —(CH₂)₂(CHR′)(CH₂)₂— or —(CH₂)₂NR′(CH₂)₂—, most preferably —(CH₂)₂(CHR′)(CH₂)₂—. An especially preferred bifunctional diaminedioxime chelator has the Formula:

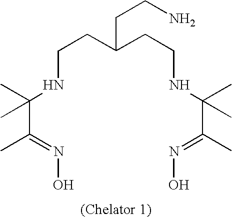

such that the tracer is conjugated via the bridgehead —CH₂CH₂NH₂ group.
(ii) N₃S ligands having a thioltriamide donor set such as MAG₃ (mercaptoacetyltriglycine) and related ligands; or having a diamidepyridinethiol donor set such as Pica;
(iii) N₂S₂ ligands having a diaminedithiol donor set such as BAT or ECD (i.e. ethylcysteinate dimer), or an amideaminedithiol donor set such as MAMA;
(iv) N₄ ligands which are open chain or macrocyclic ligands having a tetramine, amidetriamine or diamidediamine donor set, such as cyclam, monoxocyclam or dioxocyclam.
(v) N₂O₂ ligands having a diaminediphenol donor set.

The above described ligands are particularly suitable for complexing technetium eg. ^{94m}Tc or ^{99m}Tc, and are described more fully by Jurisson et al [Chem. Rev., 99, 2205-2218 (1999)]. Other suitable ligands are described in Sandoz WO 91/01144, which includes ligands which are particularly suitable for indium or yttrium, especially macrocyclic aminocarboxylate and aminophosphonic acid ligands. When the radiometal ion is technetium, the ligand is preferably a chelating agent which is tetradentate. Preferred chelating agents for technetium are: N₄ chelators having a diaminedioximes, tetramine, amidetriamine or diamidediamine donor set; N₃S chelating agents having a thioltriamide donor or diamidepyridinethiol donor set; or N₂S₂ chelating agents having a diaminedithiol donor set such as BAT or an amideaminedithiol donor set such as MAMA. Preferred such ligands include: the N₄, N₃S and N₂S₂ chelating agents described above, most preferably N₄ tetramine and N₂S₂ diaminedithiol or diamidedithiol chelating agents, especially the N₂S₂ diaminedithiol chelator known as BAT:

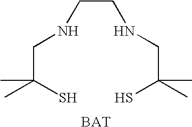

It is strongly preferred that the tracer is bound to the metal complex in such a way that the linkage does not undergo facile metabolism in blood, since that would result in the metal complex being cleaved off before the tracer reached the desired in vivo target site. The tracer is therefore preferably covalently bound to the metal complexes of the present invention via linkages which are not readily metabolised.

The term “polymer” has its conventional meaning. The polymers of the present invention may be of naturally occurring or synthetic origin, but are preferably synthetic. Suitable polymers have a molecular weight in the range 0.4 to 40 kDa preferably 1 to 10 kDa, most preferably 2 to 8 kDa. The polymers of the present invention must be sufficiently soluble in aqueous or organic solvents that the conjugate is soluble in said solvent to give the solution of step (ii) of the present method. The polymers are therefore designed to be used in solution phase chemistry, as opposed to conventional solid phase radiosynthesis. For radiofluorination, organic soluble polymers are strongly preferred because in aqueous solution the fluoride ion is too well solvated to be sufficiently reactive.

For optimal reaction conditions the greatest salvation of the solid support is required in order that the resin has as much solution-type properties as possible. A treatise on the relationship between diffusion rate and particle size is available [D. C. Sherrington “Polymer-supported Reactions in Organic Synthesis, p. 61, John Wiley and Sons Ltd, (1980)].

Suitable such solvents must also be capable of dissolving the chemical form of the radioisotope, so that the reaction of step (ii) occurs in solution. Hansen Solubility Parameters can be used to establish suitable solvent compositions that dissolve the polymer conjugates, and best solvent composition for product/polymer separation in step (iv) [Charles M. Hansen: Hansen Solubility Parameters, CRC Press (2000)]. Suitable such organic solvents include: acetonitrile, dimethylsulphoxide (DMSO), dimethylformamide (DMF), dioxane and tetrahydrofuran (THF). Most preferred such solvents are acetonitrile and DMSO. Suitable such aqueous solvents are buffer solutions or saline, especially phosphate buffered saline, phosphate buffer or borate buffer. Preferred such solvents are either aqueous or mixtures of water with water-miscible, polar organic solvents such as alcohols, acetonitrile, DMSO, DMF, THF and dioxane. Most preferred aqueous solvents are acetonitrile and DMF.

Preferred soluble polymers of the present invention are therefore chosen from:
(i) polymers soluble in organic solvents;

Macropolymeric materials such as polyethylene glycol, polyvinyl alcohol or polylysine.
(ii) polymers soluble in aqueous media;

Ficoll, polyethylenimine, Dextran and poly-L-lysine.

The following polymers are preferred:
dendrimers;
polyethylene glycol (PEG) or polypropylene glycol;
copolymers of (N-(2-hydroxypropyl)methylacrylamide) (ie. HPMA) and acrylamide-based molecules with PEG linkers are suitable;
dextran T-40 (GE Healthcare);
poly-L-lysine (Fluka);
polyvinyl alcohol (Fluka);
Chitosan (Aldrich);
polyethylenimine (Aldrich);
polyallylamine (Aldrich);
poly(dimethylamine-co-epichlorohydrin (Aldrich);
DAB-Am polypropylemimine (Aldrich); and
Ficoll PM70 (GE Healthcare).

Dendrimers are described by Inoue [Prog.Polym.Sci., 25(4), 453-571 (2000)], and Robertus et al [Rev.Mol.Biotechnol., 90(3-4), 183-193 (2002)]. Preferred dendrimers are Starburst™ PAMAM dendrimers (Aldrich).

The “biocompatible carrier medium” is a fluid, especially a liquid, in which the radioisotopically-labelled biological targeting molecule is suspended or dissolved, such that the composition is physiologically tolerable, ie. can be administered to the mammalian body without toxicity or undue discomfort. The biocompatible carrier medium is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethyleneglycols, propylene glycols and the like). The biocompatible carrier medium may also comprise biocompatible organic solvents such as ethanol. Such organic solvents are useful to solubilise more lipophilic compounds or formulations. Preferably the biocompatible carrier medium is pyrogen-free water for injection, isotonic saline or an aqueous ethanol solution. The pH of the biocompatible carrier medium for intravenous injection is suitably in the range 4.0 to 10.5.

The conjugate of step (i) is preferably of Formula I:

[polymer]-LINKER-Y-[precursor] (I)
where:
   LINKER is a bivalent organic group which spaces the reactive site (X) of the precursor from the polymer;
   Y is a group which incorporates a covalent bond which is selectively cleaved during step (iii).

The “LINKER” in the compound of Formula (I) may be any suitable organic group which serves to space (i.e. distance) the reactive site (X) of the precursor sufficiently from the polymer structure so as to maximise reactivity. Suitably, the LINKER comprises zero to four arylene groups (preferably phenylene) and/or a C₁₋₁₆ alkylene (preferably C₁₋₆ alkylene) or C₁₋₁₆ haloalkylene (preferably C₁₋₆ haloalkylene), typically C₁₋₁₆ fluoroalkylene (preferably C₁₋₆ fluoroalkylene), or C₂₋₁₆ alkoxyalkylene or C₁₋₁₆ haloalkoxy (suitably C₁₋₆ alkoxy or C₁₋₆ haloalkoxy) typically C₁₋₁₆ fluoroalkoxy (suitably C₁₋₆ fluoroalkoxy), and optionally one to four additional functional groups such as amide or sulphonamide groups.

Examples of such LINKERs are well known to those skilled in the art, and are described by Gil and Brase [Curr.Opin.Chem.Biol., 8(3), 230-237 (2004)] and James [Tetrahedron, 55(16), 4855-4946 (1999)]. Preferred such linkers include:

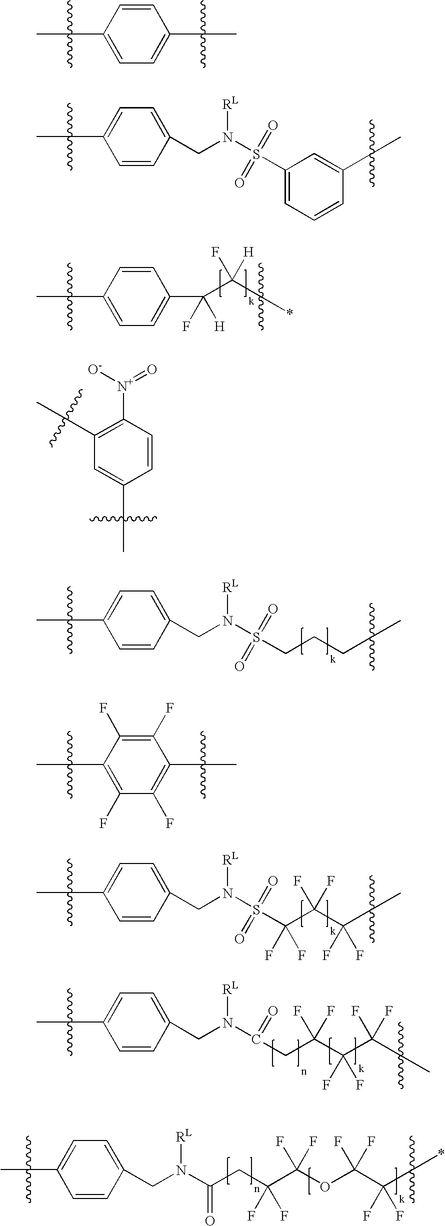

wherein at each occurrence, k is an integer of 0 to 3, n is an integer of 1 to 16, and R^{L} is H or C₁₋₆ alkyl.

Preferred alkoxy-containing LINKERs include:

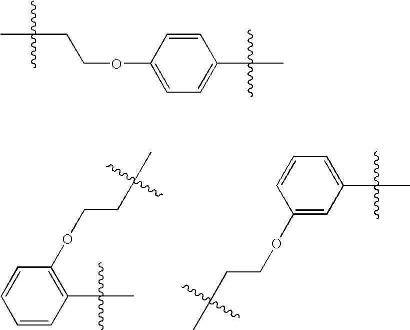

Suitable Y groups incorporating selectively cleavable covalent bonds are known in the art and include the following:
(i) acid-sensitive groups;
(ii) base-sensitive groups, such as ester linkages;
(iii) groups which can be cleaved by photochemical or thermal means;
(iv) groups which can be cleaved by electrochemical means;
(v) groups which can be cleaved by redox (oxidative or reductive) means;
(vi) groups which can be cleaved by electrophilic reaction;
(vii) groups which can be cleaved by nucleophilic substitution, such as iodonium salts;
(viii) groups which can be cleaved by enzymatic reaction.

Cleavable linker groups in organic synthesis have been reviewed by James [Tetrahedron, 55 (16), 4855-4946 (1999)]. Acid-cleavable groups include ester and imine linkages, and have been described by Floersheimer [Peptides, p131-132, (1991)] and Mergler [Tet. Lett. 29, 4005-4012 (1998)]. These are cleavable using 1% trifluoroacetic acid in a suitable solvent. Other acid labile groups have been described by Albericio [Tet. Lett. 32, 1015-1018 (1991)], and include groups which are cleavable with 0.1% trifluoroacetic acid. A similar approach to linker cleavage was employed by Rink [Tet. Lett. 28, 3787-3790 (1987)] where the labile group is cleavable in 10% acetic acid.

Base labile linkage groups have been described by Liu [Int. J. Pept. Protein Res. 35, 95-98 (1990)] together with the cleavable group described by Albericio [Tet. Lett. 32, 1515-1518 (1991)] which cleaves through a β-elimination process using piperidine or diazabicyclo-[5.4.0]undec-5-ene (DBU). A further such group is described by Garcia-Echeverria [Tet. Lett., 38(52), 8933-8934 (1997)].

Groups cleavable with fluoride ions (ie. nucleophilically) have also been developed and are described, for example by Ramage [Tetrahedron 48, 499-514 (1992)] and Mullen [Tetrahedron 28, 491-494 (1987)]. A nitrobenzophenone-based cleavable group such as that described by Findeis [J. Org. Chem. 54, 3478-3482 (1989)] and Kaiser [Science 243, 187-191 (1989)] can be cleaved nucleophilically using amines, hydrazine and carboxylic acids.

Groups which can be reductively cleaved with ammonium formate/palladium catalysed hydrogenolysis are described by Anwer [Tet. Lett., 22, 4369-4372 (1981)], whereas reductive cleavage of a 2-azidomethyl-4-hydroxy-6,N-dimethylbenzamide moiety requires triphenylphosphine [Robinson, Tetrahedron 49, 2873-2884 (1993)].

Groups which can be oxidatively cleaved are described by Arseniyadis et al [Tet. Lett., 45(10), 2251-2253 (2004)].

Groups which can be thermally cleaved are described by Keller et al [Tet. Lett., 46(7), 1181-1184 (2005)].

Selectively cleavable photolabile groups are described by Horton et al [Tet.Lett., 41(47), 9181-9184 (2000)].

By the term “chemical form of the radioisotope suitable for reaction with X” is meant a radiochemical which reacts with X in the minimum number of steps, preferably a single step to give the desired product. Preferably, the radiochemical is the form of the radioisotope which is most readily available, such as halide ions for radiohalogens or metal ions for radiometals, since it is more efficient to tailor the chemistry of the non-radioactive group X to that of the radiochemical, so as to minimise the number of radioactive steps necessary.

When the radioisotope is non-metallic, preferred convenient chemical forms of the desired non-metallic radioisotope include:
(a) halide ions (eg. ¹²³I-iodide or ¹⁸F-fluoride), especially in aqueous media, for substitution reactions;
(b) ¹¹C-methyl iodide or ¹⁸F-fluoroalkylene compounds having a good leaving group, such as bromide, mesylate or tosylate;
(c) HS(CH₂)₃¹⁸F for S-alkylation reactions with alkylating precursors such as N-chloroacetyl or N-bromoacetyl derivatives.

Preferred derivatives which undergo facile alkylation are alcohols, phenols or amine groups, especially phenols and sterically-unhindered primary or secondary amines.

Preferred X groups which alkylate thiol-containing radioisotope reactants are N-haloacetyl groups, especially N-chloroacetyl, N-bromoacetyl and N-iodoacetyl derivatives.

When the radioisotope is metallic, suitable convenient chemical forms of the radiometal are those which react readily with the ligand or chelating agent to form the desired radiometal complex. These include solution forms of the metal ion itself, especially the chemical form which would be obtained directly from a radioisotope generator (eg. ^{99m}Tc-pertechnetate); or metal complexes of the radiometal suitable for transchelation with the ligand.

After the radiolabelling of step (ii), and before the cleavage step (iii), an optional separation step may be carried out to separate the radiolabelled polymer-bound precursor from unwanted reagents, solvents or by-products of step (ii).

An especially preferred precursor is of Formula IA:

[polymer]-LINKER-Y^{X}-[precursor] (IA),
where:
   Y^{X} is a Y group which incorporates the reactive group X, and is covalently bound to the precursor by the group X so that step (iii) occurs simultaneously with the radiolabelling process of step (ii).

Suitable Y^{X} groups can be chosen from the Y groups described above, based on the “chemical form of the radioisotope suitable for reaction with X” and hence the nature of the radiolabelling reaction. Thus, eg. when radioactive halide ions (eg. ¹⁸F-fluoride or ¹²³I-iodide) are used in nucleophilic substitution, Y^{X} can be an iodonium salt, which is cleaved during the nucleophilic substitution reaction to give the desired radioisotopically-labelled imaging agent. When Y^{X} is an iodonium salt (I⁺), the LINKER preferably comprises an arylene group (most preferably phenylene) adjacent to the I⁺.

Functionalised polyethylene glycol (PEG)-based polymers containing N-hydroxysuccinimide ester, aldehyde, maleimides and mPEG-BTC (benzotriazole carbonate-mPEG) are known [Harris, “Poly(ethylene glycol) Chemistry: Biotechnical and Biomedical Applications” p1-14 Plenum Press (1992)]. Such functionalised polymers are commercially available from Polypure AS and SunBio.

Certain such functionalised polymers are suitable for use directly as conjugates of the present invention with the appropriate choice of chemical form of the radioisotope suitable for reaction with X. Thus, amine-functionalised polymers can be coupled with active ester-containing chemical form of the radioisotope, as eg. described for ¹⁸F radiolabelling above, and vice versa.

The main advantage of the Y^{X} group approach of Formula (IA) is that the radioisotopically-labelled imaging agents is not contaminated with precursor, but could potentially contain trace quantities of any protecting group(s) by-products. Since the imaging agent is generated in tracer concentrations, any such by-products would also be present in only nanomolar or picomolar concentrations, and hence would be unlikely to present any problems.

Conjugates can also be prepared using functionalised polymers as described above, plus suitable bifunctional derivatising agents. The term “bifunctional” has its conventional meaning, ie. a compound having two different types of functional group present: one comprising the precursor (and hence suitable for radiolabelling), the other suitable for conjugation with the polymer to give a covalent bond. Functional groups suitable for conjugation include: amine, thiocyanate, maleimide and active esters. Such bifunctional reagents can be reacted with suitable counterpart functional groups on the polymer to form the desired conjugate. Suitable functional groups on the polymer include:

carboxyls (for amide bond formation with an amine-functionalised bifunctional reagent);
amines (for amide bond formation with an carboxyl- or active ester-functionalised reagent);
halogens, mesylates and tosylates (for N-alkylation of an amine-functionalised reagent);
thiols (for reaction with a maleimide-functionalised reagents);
sulphonic acids (for either sulphonamide bond formation with an amine-functionalised bifunctional reagent or sulphonate ester bond formation with a hydroxyl-functionalised bifunctional reagent).

Amide coupling can be carried out directly (eg. using solid phase peptide synthesis), or in the presence of a suitable activating agent, such as BOP [ie. benzotriazol-1-yloxy-tris(dimethylamino)-phosphonium] or N,N′-dicyclohexylcarbodiimide (DCCI). The coupling can also be carried out via appropriate intermediates as is known in the art, such as activated esters of a carboxyl group. Alternatively, the pendant amine group of the bifunctional reagent can first be converted to an isothiocyanate (—NCS) or isocyanate group (—NCO) group, which permits conjugation to amine-containing compounds, via the formation of thiourea and urea linkages respectively. Alternatively, the pendant amine group of a bifunctional reagent can be reacted with a diacid to introduce a terminal carboxyl group via a linker group. A bifunctional reagent bearing a carboxyl function can be used in a similar manner to couple directly to an amine-containing molecule via an amide bond. The bifunctional reagent may also bear a group designed to react with thiol groups on the polymer to form stable thioether linkages. Examples of such groups are maleimides (which may be prepared by reaction of maleic anhydride with the corresponding amine, followed by heating with acetic anhydride), and acrylamides (which may be prepared by reaction of acrylyl chloride with the amine).

By the term “active ester” is meant an ester derivative of a carboxylic acid which is designed to be a better leaving group, and hence permit more facile reaction with nucleophiles present on the biological targeting moiety such as amines. Examples of suitable active esters are: N-hydroxysuccinimide (NHS), pentafluorophenol, pentafluorothiophenol, para-nitrophenol and hydroxybenzotriazole.

Scheme 1 shows a specific example of how conjugates of the present invention may be conveniently prepared from a sulphonic acid functionalised resin.

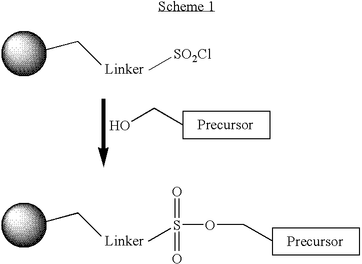

Such resins may be reacted with a chlorinating agent to give the corresponding sulphonyl chloride resin. This may be carried out by treating the resin with, for example, phosphorus pentachloride, phosphorus trichloride, oxalyl chloride, or thionyl chloride, in an appropriate inert solvent such as dichloromethane, chloroform, or acetonitrile, and heating at elevated temperature for a period of time. The excess reagent may then be removed from the resin by washing with further portions of the inert solvent. The sulphonyl chloride resin may then be reacted with an hydroxy-functionalised precursor to produce the resin-bound precursor. This may be carried out by treating the resin with a solution of the alcohol in an inert solvent such as chloroform, dichloromethane, acetonitrile, or tetrahydrofuran containing a non-nucleophilic soluble base such as sodium hydride or a trialkylamine, for example triethylamine or diisopropylethylamine. The reaction may be carried out at a temperature of 10 to 80° C., optimally at ambient temperature for a period of from around 1 to 24 hours. The excess alcohol and base may then be removed from the solid support by washing with further portions of an inert solvent such as chloroform, dichloromethane, or tetrahydrofuran.

Step (iii) of the present method, ie. cleavage from the polymer, would be carried out by conventional methods [James cited above plus Gil et al Curr.Opin.Chem.Biol., 8(3), 230-237 (2004)], in particular using selective reagents which react with the labile bond of the conjugate, but do not react with the biological targeting molecule (“tracer”). If necessary, as noted above, suitable protecting groups are used to protect the tracer.

Step (iv) of the present method, ie. separation can be achieved chromatographically or through precipitation or extraction. Suitable chromatographic methods include: C₁₈, C₈, C₄ reversed phase HPLC; ion exchange; silica; alumina; hydroxyapatite, membrane filtration, size exclusion and gel filtration. It is also envisaged that cationic (such as quaternary ammonium) or anionic (such as sulphonate) groups on the soluble polymer could aid ion exchange separation. Preferably the separation column is designed to be single-use, ie. disposable. The selection of separation method is dependent on the separation time (and hence loss of yield due to radioactive decay) as well as the efficiency of separation. Thus, for short half-life radioisotopes such as ¹⁸F (t½ 110 min), the separation time is preferably less than 15 minutes, most preferably less than 5 minutes. For longer-lived radioisotopes such as ^{99m}Tc, (t½ 6 hours), separation times of 30 to 40 minutes are feasible, but of course shorter times are preferred. Most preferably the separation column is an SPE (Solid Phase Extraction) column or a Flash Chromatography Cartridge (commercially available from a range of suppliers).

Separation can also be achieved through precipitation or extraction, using the differing solubilities of the radiolabelled imaging agent in organic and aqueous solvent. Whilst it may be possible to precipitate the radiolabelled imaging agent or the polymer, the former is preferred since no further dissolution step would be required. When the macromolecule is a protein, separation could be accomplished via heat treatment to precipitate the denatured protein. Alternatively the use of specific groups attached to the polymer, such as biotin or digoxin can be used for subsequent removal using streptavidin or anti-digoxin antibodies.

When step (vi) of the present invention includes a purification step, this could include one or more of the following:
(i) filtration to remove unwanted insoluble matter or particulates;
(ii) chromatography.

The chromatography may involve conventional normal phase or reverse phase methodology, or ion exchange methods. It suitably takes the form of HPLC, SPE or ‘flash’ chromatography cartridges. In some instances the desired product is essentially immobilised at the top of a column matrix because of much higher affinity for the stationary phase compared to the mobile phase. The impurities can thus be eluted in a mobile phase to which they have higher affinity than the stationary phase to a suitably shielded waste container. After washing, the purified product can subsequently simply be eluted using an alternative eluent system to which the product exhibits higher affinity than the stationary phase. Any such chromatography is preferably carried out using disposable columns, so that there is no risk that subsequent preparations are contaminated with material from previous preparations. Such chromatography cartridges are commercially available from a range of suppliers, including Waters and Varian.

When step (vi) of the present invention includes a pH adjustment step, this can be carried out using a pH-adjusting agent. The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], pharmaceutically acceptable acids such as acetic acid, bases and pharmaceutically acceptable bases such as sodium carbonate, sodium bicarbonate or mixtures thereof.

When steps (v) or (vi) of the present invention includes solvent removal and re-dissolution steps, the solvent can be removed by various techniques:
(i) chromatography;
(ii) application of reduced pressure or vacuum;
(iii) evaporation due to heating or bubbling of gas through or over the solution;
(iv) azeotropic distillation.

The chromatography technique applies immobilisation as described above, and is a preferred method. Such solvent removal techniques are important because they permit the preparation of the radiolabelled imaging agent by reaction in organic solvents, but the final radiopharmaceutical is still supplied in a biocompatible carrier medium. This is particularly useful for precursors or intermediates which are either poorly soluble in aqueous media or susceptible to hydrolysis in aqueous media or perhaps both. Examples of this are: trialkyltin precursors, especially tributyltin or trimethyltin derivatives. Hence, when the precursor is poorly soluble or susceptible to hydrolysis in aqueous media, the solvent used is preferably an organic solvent, most preferably a water-miscible organic solvent such as acetonitrile, ethanol, dimethylformamide (DMF), dimethylsulfoxide (DMSO) or acetone. Preferred such solvents are acetonitrile, ethanol, DMF and DMSO.

In a second aspect, the present invention provides a method of preparation of a radiopharmaceutical which comprises the radioisotopically-labelled imaging agent composition of the first aspect, said method comprising carrying out the process of the first aspect under sterile conditions and/or subjecting the product of step (vi) to terminal sterilisation, such that the product of step (vi) is in a form suitable for mammalian administration.

The method of the second embodiment may be carried out under aseptic manufacture (ie. clean room) conditions to give the desired sterile, non-pyrogenic radiopharmaceutical product. It is preferred that the key components, especially the associated reagents plus those parts of the apparatus which come into contact with the radiopharmaceutical (eg. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise the non-radioactive components in advance, so that the minimum number of manipulations need to be carried out on the radiopharmaceutical. As a precaution, however, it is preferred to include at least a sterile filtration in step (vi) of the present method.

The precursor and other such reagents and solvents are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour. The reaction vessel is suitably chosen from such containers, and preferred embodiments thereof.

The radiopharmaceutical composition products of the method of the present invention are suitably supplied in a sealed container as described above, which may contain single or multiple patient doses. Single patient doses or “unit doses” can thus be withdrawn into clinical grade syringes at various time intervals during the viable lifetime of the preparation to suit the clinical situation. Preferred multiple dose containers comprise a single bulk vial (e.g. of 10 to 30 cm³ volume) which contains sufficient radioactivity for multiple patient doses. Unit dose syringes are designed to be used with a single human patient only, and are therefore preferably disposable and suitable for human injection. The filled unit dose syringes may optionally be provided with a syringe shield to protect the operator from radioactive dose. Suitable such radiopharmaceutical syringe shields are known in the art and preferably comprise either lead or tungsten. The method of the present invention preferably further comprises sub-dispensing the radiopharmaceutical composition into unit patient doses.

The method of the second embodiment is preferably automated. Preferred automated methods are microprocessor-controlled. The term “microprocessor-controlled” has its conventional meaning. Thus, the term “microprocessor” as used herein, refers to a computer processor contained on an integrated circuit chip, such a processor may also include memory and associated circuits. The microprocessor is designed to perform arithmetic and logic operations using logic circuitry that responds to and processes the basic instructions that drive a computer. The microprocessor may also include programmed instructions to execute or control selected functions, computational methods, switching, etc. Microprocessors and associated devices are commercially available from a number of sources, including, but not limited to: Cypress Semiconductor Corporation, San Jose, Calif.; IBM Corporation; Applied Microsystems Corporation, Redmond, Wash., USA; Intel Corporation and National Semiconductor, Santa Clara, Calif. With regard to the present invention, the microprocessor provides a programmable series of reproducible steps involving eg. transfer of chemicals, heating, filtration etc.

By the term “automated synthesizer” is meant an automated module based on the principle of unit operations as described by Satyamurthy et al [Clin.Positr.Imag., 2(5), 233-253 (1999)]. The term ‘unit operations’ means that complex processes are reduced to a series of simple operations or reactions, which can be applied to a range of materials. Such automated synthesizers are preferred for the method of the present invention, and are commercially available from a range of suppliers [Satyamurthy et al, above], including GE Healthcare, CTI Inc., Ion Beam Applications S.A. (Chemin du Cyclotron 3, B-1348 Louvain-La-Neuve, Belgium), Raytest (Germany) and Bioscan (USA).

Commercial automated synthesizers also provide suitable containers for the liquid radioactive waste generated as a result of the radiopharmaceutical preparation. Automated synthesizers are not typically provided with radiation shielding, since they are designed to be employed in a suitably configured radioactive work cell. The radioactive work cell provides suitable radiation shielding to protect the operator from potential radiation dose, as well as ventilation to remove chemical and/or radioactive vapours. Suitable automated synthesizers of the present invention are those which comprise a disposable or single use cassette which comprises all the reagents, reaction vessels and apparatus necessary to carry out the preparation of a given batch of radiolabelled radiopharmaceutical. Such cassettes are described in the fifth embodiment below. The cassette means that the automated synthesizer has the flexibility to be capable of making a variety of different radioisotope-labelled radiopharmaceuticals with minimal risk of cross-contamination, by simply changing the cassette. The cassette approach has the advantages of: simplified set-up hence reduced risk of operator error; GMP compliance; multi-tracer capability; rapid change between production runs; pre-run automated diagnostic checking of the cassette and reagents; automated barcode cross-check of chemical reagents vs the synthesis to be carried out; reagent traceability; single-use and hence no risk of cross-contamination, tamper and abuse resistance. As noted above, the cassette approach is also versatile so overcomes the prior art problem of having to redesign a whole new automated synthesis apparatus each time a different radiopharmaceutical is to be prepared.

In a third aspect, the present invention provides a precursor suitable for use in the methods of the first and second embodiments. The precursor and preferred embodiments thereof are as described in the first embodiment, above.

In a fourth aspect, the present invention provides a kit which comprises the precursor of the third aspect. Such kits are non-radioactive. When the radioisotope is a radiometal, suitable kits comprise the [ligand]-[polymer] conjugate, including preferred aspects thereof, as described in the first embodiment above. When the radiometal is ^{99m}Tc, the kit suitably further comprises a biocompatible reductant.

Such kits are particularly useful in the preparation of radiopharmaceuticals, ie. in the method of the second embodiment. Such radiopharmaceutical kits are designed to give sterile products suitable for human administration, e.g. via direct injection into the bloodstream. Such kits are preferably lyophilised and is designed to be reconstituted with sterile supply of the radioisotope, with the minimum of additional steps. For ^{99m}Tc, ^{99m}Tc-pertechnetate (TcO₄⁻) from a ^{99m}Tc radioisotope generator to give a solution suitable for human administration without further manipulation. Suitable kits comprise a container (eg. a septum-sealed vial) containing the ligand or chelator conjugate in either free base or acid salt form, together with a biocompatible reductant such as sodium dithionite, sodium bisulphite, ascorbic acid, formamidine sulphinic acid, stannous ion, Fe(II) or Cu(I). The biocompatible reductant is preferably a stannous salt such as stannous chloride or stannous tartrate. Alternatively, the kit may optionally contain a metal complex which, upon addition of the radiometal, undergoes transmetallation (i.e. metal exchange) giving the desired product.

The non-radioactive kits may optionally further comprise additional components such as a radioprotectant, antimicrobial preservative, pH-adjusting agent, filler or transchelator. By the term “radioprotectant” is meant a compound which inhibits degradation reactions, such as redox processes, by trapping highly-reactive free radicals, such as oxygen-containing free radicals arising from the radiolysis of water. The radioprotectants of the present invention are suitably chosen from: ascorbic acid, para-aminobenzoic acid (ie. 4-aminobenzoic acid), gentisic acid (ie. 2,5-dihydroxybenzoic acid) and salts thereof with a biocompatible cation. By the term “biocompatible cation” is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

By the term “antimicrobial preservative” is meant an agent which inhibits the growth of potentially harmful micro-organisms such as bacteria, yeasts or moulds. The antimicrobial preservative may also exhibit some bactericidal properties, depending on the dose. The main role of the antimicrobial preservative(s) of the present invention is to inhibit the growth of any such micro-organism in the radiopharmaceutical composition post-reconstitution, ie. in the radioactive diagnostic product itself. The antimicrobial preservative may, however, also optionally be used to inhibit the growth of potentially harmful micro-organisms in one or more components of the non-radioactive kit of the present invention prior to reconstitution. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the reconstituted kit is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethane], and pharmaceutically acceptable bases such as sodium carbonate, sodium bicarbonate or mixtures thereof. When the conjugate is employed in acid salt form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

By the term “transchelator” is meant a compound which reacts rapidly to form a weak complex with technetium, then is displaced by the ligand. This minimises the risk of formation of reduced hydrolysed technetium (RHT) due to rapid reduction of pertechnetate competing with technetium complexation. Suitable such transchelators are salts of a weak organic acid, ie. an organic acid having a pKa in the range 3 to 7, with a biocompatible cation. Suitable such weak organic acids are acetic acid, citric acid, tartaric acid, gluconic acid, glucoheptonic acid, benzoic acid, phenols or phosphonic acids. Hence, suitable salts are acetates, citrates, tartrates, gluconates, glucoheptonates, benzoates, phenolates or phosphonates. Preferred such salts are tartrates, gluconates, glucoheptonates, benzoates, or phosphonates, most preferably phosphonates, most especially diphosphonates. A preferred such transchelator is a salt of MDP, ie. methylenediphosphonic acid, with a biocompatible cation.

In a fifth aspect, the present invention provides a single use cassette suitable for use in the radiopharmaceutical preparation method of the second embodiment, especially an automated such method. By the term “cassette” is meant a piece of apparatus designed to fit removably and interchangeably onto an automated synthesizer apparatus (as defined above), in such a way that mechanical movement of moving parts of the synthesizer controls the operation of the cassette from outside the cassette, ie. externally. Suitable cassettes comprise a linear array of valves, each linked to a port where reagents or vials can be attached, by either needle puncture of an inverted septum-sealed vial, or by gas-tight, marrying joints. Each valve has a male-female joint which interfaces with a corresponding moving arm of the automated synthesizer. External rotation of the arm thus controls the opening or closing of the valve when the cassette is attached to the automated synthesizer. Additional moving parts of the automated synthesizer are designed to clip onto syringe plunger tips, and thus raise or depress syringe barrels.

The cassette is versatile, typically having several positions where reagents can be attached, and several suitable for attachment of syringe vials of reagents or chromatography cartridges (eg. SPE). The cassette always comprises a reaction vessel. Such reaction vessels are preferably 1 to 10 cm³, most preferably 2 to 5 cm³ in volume and are configured such that 3 or more ports of the cassette are connected thereto, to permit transfer of reagents or solvents from various ports on the cassette. Preferably the cassette has 15 to 40 valves in a linear array, most preferably 20 to 30, with 25 being especially preferred. The valves of the cassette are preferably each identical, and most preferably are 3-way valves. The cassettes of the present invention, are designed to be suitable for radiopharmaceutical manufacture and are therefore manufactured from materials which are of pharmaceutical grade and ideally also are resistant to radiolysis.

In a sixth aspect, the present invention provides the use of an automated synthesizer apparatus which is adapted to accept the cassette of the fifth embodiment, for carrying out the preferred automated radiopharmaceutical preparation method of the second embodiment. The “automated synthesizer” is as defined for the second embodiment above, such that it interfaces with the interchangeable, single use cassette of the fifth embodiment. The automated synthesizer is preferably used to carry out the radiopharmaceutical preparation via the method of the first embodiment, including preferred embodiments thereof.

In a seventh aspect, the present invention provides the use of the cassette of the third embodiment for carrying out the preferred automated radiopharmaceutical preparation method of the second embodiment. The method and radiopharmaceutical, plus preferred embodiments thereof are as described in the first embodiment. The cassette and preferred embodiments thereof are as described in the third embodiment.

The invention is illustrated by the following non-limiting Example.

Abbreviations used.
PEG=polyethyleneglycol;
PG=protecting group;
PVA=polyvinylalcohol;
PVP=poly(vinylpyrrolidone);
TFA=trifluoroacetic acid.

### EXAMPLE 1

### Synthesis of ¹⁸F-DOPA

This is a prophetic Example.

The approach which would be used is given in FIG. 1:

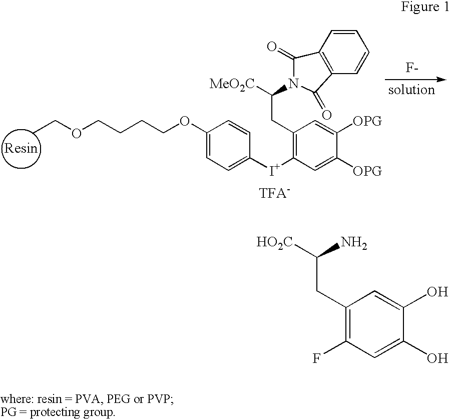

The iodonium salts would be prepared by the methods of Pike et al [JCS Perkin Trans., 2043 (1998)] and as described in WO 2004/056400. The DOPA precursors can be obtained as described by Bolton [J.Lab.Comp.Radiopharm., 45, 485-528 (2002)].

What is claimed is:

1. A method of preparation of a radioisotopically-labelled imaging agent composition which comprises the process of:
(i) provision of a conjugate which comprises a precursor to said imaging agent covalently bound to a polymer, wherein said precursor has at least one group (X) which provides a reactive site for radiolabelling;
(ii) reaction in a suitable solvent of a solution of the conjugate from step (i) with a chemical form of the radioisotope suitable for reaction with X to give a solution of the radiolabelled precursor bound to said polymer;
(iii) cleavage of the radiolabelled precursor product of step (ii) from the polymer;
(iv) separation of the cleaved radiolabelled precursor product of step
(iii) from the polymer and optionally from other reaction products of steps (ii) and (iii);
(v) when the separated radiolabelled precursor product of step (iv) is already in a biocompatible carrier medium, it is used directly in step (vi), otherwise the product of step (iv) is either dissolved in a biocompatible carrier medium or the solvent of step (iv) is removed in part or in full, and replaced with a biocompatible carrier medium;
(vi) optionally carrying out one or more of the following additional processes on the product of step (v): purification; pH adjustment; dilution or concentration; solvent removal and re-dissolution in a biocompatible solvent; to give the desired imaging agent composition.

2. The method of claim 1, where the polymer of the conjugate has a molecular weight in the range 400 Daltons to 40 k Daltons.

3. The method of claim 1, where the conjugate of step (i) is of Formula I:
[polymer]-LINKER-Y-[precursor] (I)
where:
LINKER is a bivalent organic group which spaces the reactive site (X) of the precursor from the polymer;
Y is a group which incorporates a covalent bond which is selectively cleaved during step (iii).

4. The method of claim 3, where the precursor is of Formula IA:
[polymer]-LINKER-Y^{X}-[precursor] (IA),
where:
Y^{X} is a Y group which incorporates the reactive group X, and is covalently bound to the precursor by the group X so that step (iii) occurs simultaneously with the radiolabelling process of step (ii).

5. The method of claim 1, where the radioisotope is a positron emitter.

6. The method of claim 5, where the positron emitter is chosen from ¹⁸F, ¹¹C, ¹⁵N, or ¹⁸O.

7. The method of claim 1, where the radioisotope is ¹⁸F and the chemical form of the radioisotope suitable for reaction with X is ¹⁸F-fluoride.

8. The method of claim 1, where the solvent used in step (ii) is an organic solvent.

9. The method of claim 1, where the solvent used in step (ii) is an aqueous solvent.

10. A method of preparation of a radiopharmaceutical which comprises the radioisotopically-labelled imaging agent composition of claim 1, said method comprising carrying out the process of said claim under sterile conditions and/or subjecting the product of step (vi) to terminal sterilisation, such that the product of step (vi) is in a form suitable for mammalian administration.

11. The method of claim 10, wherein the process is automated and comprises:
(i) provision of a conjugate which comprises a precursor to said imaging agent covalently bound to a polymer, wherein said precursor has at least one group (X) which provides a reactive site for radiolabelling;
(ii) reaction in a suitable solvent of a solution of the conjugate from step (i) with a chemical form of the radioisotope suitable for reaction with X to give a solution of the radiolabelled precursor bound to said polymer;
(iii) cleavage of the radiolabelled precursor product of step (ii) from the polymer;
(iv) separation of the cleaved radiolabelled precursor product of step (iii) from the polymer and optionally from other reaction products of steps (ii) and (iii);
(v) when the separated radiolabelled precursor product of step (iv) is already in a biocompatible carrier medium, it is used directly in step (vi), otherwise the product of step (iv) is either dissolved in a biocompatible carrier medium or the solvent of step (iv) is removed in part or in full, and replaced with a biocompatible carrier medium;
(vi) optionally carrying out one or more of the following additional processes on the product of step (v): purification; pH adjustment; dilution or concentration; solvent removal and re-dissolution in a biocompatible solvent; to give the desired imaging agent composition.

12. The method of claim 11, wherein an automated synthesizer apparatus is used to automate the process.

13. The method of claim 12, where the automated synthesizer apparatus comprises a single use cassette, wherein said cassette comprises non-radioactive reagents necessary to carry out the process of
(i) provision of a conjugate which comprises a precursor to said imaging agent covalently bound to a polymer, wherein said precursor has at least one group (X) which provides a reactive site for radiolabelling;
(ii) reaction in a suitable solvent of a solution of the conjugate from step (i) with a chemical form of the radioisotope suitable for reaction with X to give a solution of the radiolabelled precursor bound to said polymer;
(iii) cleavage of the radiolabelled precursor product of step (ii) from the polymer;
(iv) separation of the cleaved radiolabelled precursor product of step (iii) from the polymer and optionally from other reaction products of steps (ii) and (iii);
(v) when the separated radiolabelled precursor product of step (iv) is already in a biocompatible carrier medium, it is used directly in step (vi), otherwise the product of step (iv) is either dissolved in a biocompatible carrier medium or the solvent of step (iv) is removed in part or in full, and replaced with a biocompatible carrier medium;
(vi) optionally carrying out one or more of the following additional processes on the product of step (v): purification; pH adjustment; dilution or concentration; solvent removal and re-dissolution in a biocompatible solvent; to give the desired imaging agent composition.

14. The method of claim 13, where the cassette components and reagents are in sterile, apyrogenic form.

15. A precursor suitable for use in the methods of claim 1, wherein said precursor is as defined in said claim.

16. A kit suitable for use in the method of claim 1 which comprises the precursor of said claim.

17. A single use cassette suitable for use in the method claim 10, wherein said cassette comprises non-radioactive reagents.

18. Use of an automated synthesizer apparatus to automate a process for carrying out the process of claim 11.

19. Use of a single cassette in the automated synthesizer apparatus wherein said cassette comprises non-radioactive reagents for carrying out the process of claim 11.
